(19) United States
(12) Patent Application Publication
(10) Pub. No.: US 2005/0196751 A1
(43) Pub. Date: Sep. 8, 2005

(54) Adenoviral vector-mediated delivery of modified steroid hormone receptors and related products and methods

(76) Inventors: Mark M. Burcin, (US); Bert W. O'Malley, (US); Gudrun Schiedner, (US); Sophia Y. Tsai, (US); Stefan Kochanek, (US)

Correspondence Address: PERKINS COIE LLP, POST OFFICE BOX 1208, SEATTLE, WA, 98111-1208, US

(21) Appl. No.: 09/861,181

(22) Filed: May 18, 2001

Related U.S. Application Data
(63) Continuation of application No. PCT/US99/26802, filed on Nov. 12, 1999.
(60) Provisional application No. 60/109,185, filed on Nov. 20, 1998.

(51) Int. Cl. C12Q 1/68; C07H 21/04; A61K 48/00; C07K 14/705

(52) U.S. Cl. 435/6; 435/69.1; 435/456; 435/320.1; 435/325; 530/350; 536/23.2; 424/93.2

(57) ABSTRACT

The present invention relates to adenoviral delivery of modified steroid hormone receptor proteins. The adenoviral vector preferably contains no viral coding sequence and is capable of accepting a large insert. Such vectors preferably are capable of achieving high levels and durations of delivery and expression. The modified protein preferably is capable of distinguishing a hormone agonist from an antagonist and may be modified in the ligand binding domain, the DNA binding domain, and/or the transregulatory domain.

### PRIORITY CLAIM

The present application claims priority to PCT Application Serial No. PCT/US99/26802, filed Nov. 12, 1999, which, in turn, claims priority to U.S. Provisional Application Ser. No. 60/109,185, filed Nov. 20, 1998. Both the PCT Application and the Provisional Application are hereby incorporated by reference as if fully set forth herein.

### INTRODUCTION

The present invention relates generally to gene transfer and modified steroid hormone receptors, including molecular switches, for gene therapy. More specifically, the present invention relates to novel strategies for adenoviral vector-mediated gene transfer of modified steroid hormone receptors and related products and methods.

### BACKGROUND OF THE INVENTION

The following description of the background of the invention is intended to aid in the understanding of the invention, but is not admitted to describe or constitute prior art to the invention.

Modified steroid hormones, including molecular switches and mutated steroid hormones, for gene therapy and methods for their use have previously been described, for example in: (1) “Modified Steroid Hormones for Gene Therapy and Methods for Their Use” International Patent Publication No. WO 96/40911 (PCT/US96/0432); (2) “Mutated Steroid Hormone Receptors, Methods for Their Use and Molecular Switch for Gene Therapy” International Patent Publication No. WO 93/23431 (PCT/US93/0439), published Nov. 25, 1993; (3) “Mutated Progesterone Receptors and Methods for Their Use”, U.S. Pat. No. 5,3564,791, issued Nov. 15, 1994; and (4) “Modified Steroid Hormones for Gene Therapy and Methods for Their Use”, U.S. patent application Ser. No. 08/959,013, filed Oct. 28, 1997, all of which are incorporated herein by reference in their entirety, including any drawings.

Modified steroid hormones generally include three domains: (1) a DNA binding domain, (2) a ligand binding domain and (3) a transregulatory domain. There are several specific examples of the use of this technology. For example: (1) the positive and negative regulation of gene expression in eukaryotic cells with an inducible transcriptional regulator is described in Wang, et al., Gene Therapy, 4:432-441, 1997; (2) drug inducible transgene expression in brain using a herpes simplex virus vector is described in Oligino, et al., Gene Therapy, 5:491-496, 1998; and (3) ligand-inducible and liver specific target gene expression in transgenic mice is described in Wang, et al., Nature Biotechnology, 15:239-243, 1997, all of which are incorporated herein by reference in its entirety including any drawings.

Several methods, primarily utilizing non-viral technology have thus been used to deliver modified steroid hormones in the past. Viral delivery of such products has been suggested (for example, see “Mutated Steroid Hormone Receptors, Methods for Their Use and Molecular Switch for Gene Therapy”, U.S. patent application Ser. No. 08/479,846, filed Jun. 6, 1995, which is incorporated herein by reference in its entirety, including any drawings), however delivery via an adenoviral vector (such as the one described in Morsy, et al., Proc. Nat'l. Acad. Sci. USA, 95:7866-7871, 1998, which is incorporated herein by reference in its entirety including any drawings) has not previously been described.

Thus, despite the recent and significant advances in non-viral delivery of modified steroid, there remains a need in the art for additional means of delivery for such products.

### SUMMARY OF INVENTION

The present invention relates to novel adenoviral vector delivery of modified steroid hormone receptors and related products and methods.

The present invention thus provides adenoviral vectors which contain coding sequences for modified steroid hormone receptor proteins. Any modified steroid hormone receptor protein may be used in accordance with the present invention. Thus, the steroid hormone receptor protein may have been modified at the ligand binding domain, so that the receptor protein is able to recognize non-natural ligands, anti-hormones, and non-native ligands. Steroid hormone receptor proteins which have been modified at the DNA binding domain are also disclosed. Also, any of the modified steroid hormone receptors may contain a transactivation domain, either with or without modification.

The present invention also provides for an insulator sequence which may be included in the adenoviral vector. Also disclosed are transgenic animals and tranfected cells which contain the coding sequence for any of the adenoviral vectors of the invention. Methods of regulating the expression of a nucleic acid cassette in gene therapy by using adenoviral vectors to transfect cells of or in an animal, preferably a mammal, most preferably a human, with the coding sequence for modified steroid hormone receptor proteins are also provided. The present invention also features methods of gene therapy using the adenoviral vectors for treating disorders such as arthritis, asthma, senile dementia and Parkinson's disease.

The adenoviral vector used to deliver the modified protein can be any conventional adenoviral vector, but preferably has no viral coding sequence and is capable of accepting a large insert, such as the vector described in Morsy, et al., Proc. Nat'l Acad. Sci. USA, 95:7866-7871, 1998, which is incorporated herein by reference in its entirety including any drawings

Definitions for many terms below are provided in (1) “Modified Steroid Hormones for Gene Therapy and Methods for Their Use” International Patent Publication No. WO 96/40911 (PCT/US96/0432); (2) “Mutated Steroid Hormone Receptors, Methods for Their Use and Molecular Switch for Gene Therapy” International Patent Publication No. WO 93/23431 (PCT/US93/0439), published Nov. 25, 1993; (3) “Mutated Progesterone Receptors and Methods for Their Use”, U.S. Pat. No. 5,3564,791, issued Nov. 15, 1994; and (4) “Modified Steroid Hormones for Gene Therapy and Methods for Their Use”, U.S. patent application Ser. No. 08/959,013, filed Oct. 28, 1997, all of which are incorporated herein by reference in their entirety, including any drawings.

Thus, in one aspect, the present invention provides an adenoviral vector which contains a coding sequence for a modified steroid hormone receptor protein. The adenoviral vector may be capable of accepting a large insert (preferably up to 15 kb, more preferably up to 25 kb most preferably up to 35 kb or about 35 kb), does not encode viral proteins, and/or contains an insulator sequence.

The receptor protein coded for is capable of distinguishing a hormone antagonist from an agonist. Preferably, the receptor protein activates transcription of a desired gene (such as a gene encoding human growth hormone) when in the presence of an agonist for the receptor protein and when bound to an antagonist for the receptor protein.

The receptor protein preferably has a modified progesterone receptor ligand binding domain, a GAL-4 DNA binding domain, and/or a VP 16 or p65 transregulatory domain.

The modified steroid hormone ligand binding domain of the receptor protein may be modified by deletion of carboxy terminal amino acids, preferably, from about one to one hundred-twenty carboxy terminal amino acids are deleted, more preferably, from about one to about sixty carboxy terminal amino acids are deleted, most preferably, forty-two carboxy terminal amino acids are deleted.

The modified steroid hormone receptor protein may contain a modified ligand binding domain. The modified ligand binding domain may be modified by deletion of from about two to about five carboxy terminal amino acids from the ligand binding domain. The modified steroid hormone receptor protein may also be a progesterone receptor (hereinafter referred to as “PR”) with the ligand binding domain replaced with a modified ligand binding domain which binds non-natural or non-native ligands.

In one embodiment, the modified ligand binding domain of the modified steroid hormone receptor protein is modified to bind a compound which is a non-natural ligand (e.g., RU486), an anti-hormone, a non-native ligand, or a compound which is selected from the following group: 5-alpha-pregnane-3,2-dione; 11β-(4-dimethylaminophenyl)-17β-hydroxy-17α-propinyl-4,9-estradiene-3-one; 11β-(4-dimethylaminophenyl)-17α-hydroxy-17β-(3-hydroxypropyl)-13α-methyl-4,9-gonadiene-3-one; 11β-(4-acetylphenlyl)-17β-hydroxy-17α-(1-propinyl)-4,9-estradiene-3-one; 11β-(4-dimethylaminophenyl)-17β-hydroxy-17α-(3-hydroxy-1(Z)-propenyl-estra-4,9-diene-3-one; (7β, 11β, 17β)-11-(4-dimethylaminophenyl)-7-methyl-4′,5′-dihydrospiro[ester-4,9-diene-17,2′(3′H)-furan]-3-one; (11β, 14β, 17α)-4′,5′-dihydro-11-(4-dimethylaminophenyl)-[spiroestra-4,9-diene-17,2′(3′H)-furan]-3-one.

The modified steroid hormone receptor protein may contain a non-native or modified DNA binding domain, preferably GAL-4 DNA, virus DNA binding site, insect DNA binding site, or a non-mammalian DNA binding site. The modified steroid hormone receptor protein may also contain a transactivation domain, preferably VP-16, TAF-1, TAF-2, TAU-1, or TAU-2 linked to the modified steroid receptor.

The modified steroid hormone receptor may also be tissue specific. The tissue specificity of the modified steroid hormone receptor may be determined by adding a transactivation domain which is specific to a given tissue. The tissue specificity may also be determined by the ligand which binds to the modified steroid hormone receptor. The modified steroid hormone receptor may also contain a tissue specific element to the target gene.

The vector may contain a coding sequence for a modified steroid hormone receptor, for example a modified glucocorticoid receptor protein, for regulating expression of a promoter transcriptionally linked to nucleic acid encoding a desired protein. The modified steroid hormone receptor contains a DNA binding domain which binds the promoter, a transactivation domain which causes transcription from the promoter when the modified steroid hormone receptor is bound to the promoter and to an agonist for the modified steroid hormone receptor. The modified steroid hormone receptor may also contain a modified steroid hormone superfamily receptor ligand binding domain distinct from a naturally occurring steroid hormone superfamily receptor ligand binding domain, in that when it is bound to an agonist for the naturally occurring steroid hormone superfamily receptor, the modified steroid hormone receptor activates the transactivation domain to cause the transcription of the nucleic acid.

The vector may also be capable of regulating expression of a nucleic acid cassette in a transgenic animal or in a plant. Thus, the invention provides a method for regulating expression of a nucleic acid cassette in gene therapy which includes the steps of attaching the coding sequence of any of the modified steroid receptor proteins discussed herein to a nucleic acid cassette to form a nucleic acid cassette/modified steroid receptor protein complex, and inserting of the complex into an adenoviral vector. In one embodiment, the method includes the step of administering a pharmacological dose of the adenoviral vector to an animal or human to be treated. The nucleic acid cassette and the modified steroid hormone receptor protein may be in a positional relationship so that the expression of the nucleic acid sequence in the nucleic acid cassette is capable of being up-regulated or down-regulated by the modified steroid receptor protein.

The method may also be used for treating arthritis, asthma, senile dementia or Parkinson's disease. In this case, the nucleic acid cassette contains the nucleic acid sequence coding for a protein, such as a glucocorticoid receptor protein, a hormone, or a neurotransmitter and a growth factor. The method may also include the step of encapsulating a transformed cell, preferably a brain cell, which contains the nucleic acid cassette/modified steroid hormone receptor complex in a permeable structure. The permeable structure preferably is capable of allowing the passage of activators of the modified steroid hormone receptor protein translated from the nucleic acid sequence, but prevents the passage of attack cells.

The method of the present invention can be used to treat a variety of acquired and inherited diseases. One skilled in the art will be able to identify the proper therapeutic gene to insert into the vector depending on the disease or condition. Disease ammenable to treatment include but are not limited to growth hormone insufficency and aging disorders by selecting the growth hormone gene, obesity by selecting the leptin gene, low hematocrit by selecting the EPO gene, low vascularization of cardiac or peripheral muscle by selecting any of the VEGF genes or FGF genes, hypercholesterolemia by selecting the LDL receptor gene or the VLDL receptor gene, hemophelia by selecting the Factor VIII or the Factor IX gene, and cancer including metastatic cancer by selecting an interleukin gene or an antiangiogenic gene such as endostatin or angiostatin. Multiple genes may be incorporated into the vector to treat diseases or conditions were a complex pathway or disease state exists.

“Treat” or treatment” means to improve an animal or human suffering from a disease toward a more normal state. Treatment does not necessarily imply or suggest a cure. Treatment is simply making the diseased animal or human more normal. Treatment of hemophelia for example may be by elevating the circulating levels of an abarrent or missing clotting factor by 0.01, 0.1 or 1% of the pretreatment level.

The method may also be practiced so that the nucleic acid sequence is transcribed to produce a protein after the animal or human is given a pharmacological dose of, for example, an anti-progesterone. The amount of protein produced in the transformed cell may be proportional to the dose of anti-progesterone. The coding sequence for the modified steroid hormone receptor and the nucleic acid cassette may be in separate adenoviral vectors and may be co-injected into a target cell or animal. The regulation may also be the transactivation of glucocorticoid responsive genes or the transrepression of NF-κB and AP-1 regulated genes.

In a further aspect, a transgenic animal is provided whose cells contain any of the adenoviral vectors discussed herein. A transfected cell is also provided which contains DNA which codes for any of the modified steroid hormone receptor proteins discussed herein. In various embodiments, the cell may be a yeast, mammalian, or insect cell. The transfected cell may be the yeast Saccharomyces cerevisiae, a mammalian cell (preferably a HeLa, CV-1, COSM6, HepG2, CHO or Ros 17.2 cell), an insect cell (preferably an SF9, Drosophila, butterfly or bee cell). The invention also provides a method of making a transformed cell in situ which includes the step of contacting the cell with any of the adenoviral vectors discussed herein for a time sufficient to transform the cell. The transformed cell preferably expresses a modified receptor protein encoded by the vector.

In another aspect, a method is provided of using a modified steroid receptor protein which includes the step of transforming a cell with any of the adenoviral vectors discussed herein. The transformed cells express the modified steroid receptor protein and the modified steroid receptor protein is capable of regulating expression of steroid responsive genes by binding a non-natural ligand. In other embodiments of this method, the transformed cell may be a muscle cell, lung cell, or a synovial cell.

The present invention also provides a composition of matter which contains a coding sequence for any of the modified steroid hormone receptor proteins discussed above, which are linked to a nucleic acid cassette. The coding sequence and the nucleic acid cassette are contained in an adenoviral vector. The cassette/modified steroid hormone receptor complex, is positionally and sequentially oriented in the vector so that the nucleic acid in the cassette can be transcribed and, when necessary, translated in a target cell. In other embodiments, the compositions of matter may contain a promoter which contains steroid response elements.

The summary of the invention described above is non-limiting and other features and advantages of the invention will be apparent from the following description of the preferred embodiments, and from the claims.

### BRIEF DESCRIPTION OF DRAWINGS

FIG. 1A shows a regulatory system. The regulator GLVPc consists of a mutated human progesterone ligand binding domain (hPR-LBD), a DNA binding domain of yeast GAL4 (GAL4-DBD) and an activation domain of herpes simplex virus (VP-16). Regulator GLp65 contains the activation domain of p65 derived from human NF-κB. The target consists of our GAL4 binding sites and a TATA-box linked to the luciferase reporter gene.

FIG. 1B shows RU486-dependent target-gene induction by GLVPc compared to GLp65. GLVPc or GLp65 (0.3 μg per well on a 6-well plate) were transiently transfected in Hela cells with the 17x4-TATA-luciferase as a reporter (0.3 μg per well on a 6-well plate).

FIG. 1C also shows RU486-dependent target-gene induction by GLVPc compared to GLp65. GLVPc or GLp65 (0.3 μg per well on a 6-well plate) were transiently transfected in Hela cells, but using 17x4-tk-luciferase as a reporter. The luciferase activity is shown as relative luciferase units (RLU). Control=transfection of the reporter and expression vector backbone. (+)=Presence of RU486 [10-8], (−)=absence of RU486. Error bars show standard deviation.

FIG. 2 shows the structure of hGH-GLp65 and hGH-H-GLp65. The constructs contain: The left terminus of adenovirus type 5 (hereinafter referred to as “Ad5”)(nt 1-440), a 16054 bp fragment of the human hypoxanthine-guanine phophoribosyltransferase (herein after referred to as “HPRT”) gene, a regulatory cassette containing; UAS-TATA-GH=human growth hormone under UAS-TATA control; 2×HS4=Insulator, a 5′element of the chickenβ-globin domain; TTRB=Liver specific promoter enhancer; GLp65=inducible gene switch p65 activation domain; SV40=poly A, the 6,545 bp fragment out of the C346 cosmid and the right terminus of adenovirus type 5 (nt 35818-35935). HGH-H-GLp65 contains an insulator sequence; hGH-GLp65 does not.

FIG. 3 shows induction of hGH upon adenoviral transduction. FIG. 3A shows that C57BL/6 mice (8-10 weeks) were infected in the tail vein at day 0 with 1×10⁹ infectious particle units (IU) of hGH-GLp65. RU486 (250 μg/kg) was administered every second day after infection for a period of two weeks as indicated by arrows. Mice were bled at different time points and serum hGH was analyzed by radio-immunoassay. Mice 1 and 2 received intraperitoneal injections (IP) of RU486; Mouse 3 (−RU486) received sesame oil. hGH serum levels are shown as μg/ml.

FIG. 3B shows the kinetics of inducing hGH in mice two weeks after adenoviral infection. Mice infected for two weeks with the regulatable adenoviral construct hGHGLp65 were induced with 500 mg/kg RU486 as indicated by an arrow. 3, 6, 12, 24, 48, 72, 120 and 192 hours after RU486 administration blood was drawn from the mice, and hGH was measured in the serum by a radio-immunoassay. hGH serum levels of individual mice are shown in μg/ml.

FIG. 4 shows repetitive induction of hGH in transduced mice. Mice infected with hGH-GLp65 or hGH-H-GLp65 adenoviral vectors were induced 3 times with 250 μg/kg RU486 over a time period of 50 Days. hGH was measured prior to, 12 hours after, and 7 days after RU486 administration. Graph shows independent mice that received RU486 (+) or just carrier as a control (−). Serum levels of hGH are shown in μg/ml.

FIG. 5 shows long-term expression of hGH in transduced mice. Mice infected with hGH-GLp65 or hGH-H-GLp65 received 4 weeks after infection biodegradable pellets (360 μg/pellet, released in 60 days) by transplantation containing RU486 (+) or carrier (−) only. Mice were weighed and blood was drawn 3, 13, 20 and 27 days after drug administration. FIG. 5A shows hGH levels (μg/ml). The numbers of mice for each construct is 3; bars show the standard error. FIG. 5B shows the weight of the mice (g).

FIG. 6 shows adenoviral mediated inducible hGH expression in hepatocytes. The viral construct without insulator (GLp65) was compared with the construct containing the insulator (GLp65+HS4). 2×10⁵ cells were infected with 1×10⁹ viral particles. Three hours after infection the media was changed and RU486 at a concentration of 10⁻⁸ was added. 24 hours later hGH was monitored using a radio-immunoassay. The figure displays the amounts of hGH in ng/ml cell media. GLp65=hGH-GLp65, GLp65+HS4=hGH-H-GLp65, n.d.=no detectable levels of hGH.

FIG. 7 shows the structure of the STK-GH-H-GLp65 construct. In a cell, such as a liver cell the TTRB promoter is “on” and expresses GLp65. Upon addition of RU486, GLp65 dimerizes and enters the nucleus and causes expression of growth hormone.

FIG. 8 shows the structure of the STK-GH-GLp65 5V construct.

FIG. 9 shows the results of adenoviral mediated inducible hGH expression in hepatocytes.

FIG. 10 shows inducible hGH expression in mice transduced with the STK-GH-H-GLp65 adenovival vector.

FIG. 11 shows the kinetics of hGH expression in vivo.

The drawings are not necessarily to scale. Certain features of the invention may be exaggerated in scale or shown in schematic form in the interest of clarity and conciseness.

### DETAILED DESCRIPTION OF THE PREFERRED EMBODIMENT

The present invention relates to adenoviral delivery of modified steroid hormone receptor proteins. The adenoviral vector preferably contains no viral coding sequence and is capable of accepting a large insert. Such vectors preferably are capable of achieving high levels and durations of delivery and expression. The modified protein preferably is capable of distinguishing a hormone agonist from an antagonist and may be modified in the ligand binding domain, the DNA binding domain, and/or the transregulatory domain. Those skilled in the art will be able to construct vectors of the invention using techniques and methodology already available in the art. Indeed, since suitable modified proteins and adenoviral vectors have already been described in detail separately in the past, it will be routine for one in the art to combine these two components given the teachings herein. In addition, several conventional uses of nucleic acid vectors, as described in detail herein, can be readily adapted by utilizing instead the novel vectors of the present invention.

In order to regulate expression of a transferred gene in response to an exogenous compound, we have combined a high capacity adenoviral vector devoid of all viral coding sequences with a regulatory system which can be used to express a target gene in vivo in a selected site and at a desired time. This system utilizes a novel chimeric transactivator, GLp65, which consists of a modified progesterone receptor ligand-binding domain fused to the GAL4 DNA binding domain and part of the activation domain of the human p65 protein, a component of the NF-κB complex. In the presence of the anti-progestin mifepristone (RU486), this chimeric regulator binds to a target gene containing the 17-mer GAL4 binding site, resulting in an efficient ligand-inducible transactivation of the target gene.

We inserted the novel regulator GLp65 and a regulatable human growth hormone (hGH) target gene containing the 17mer GAL4 binding site into the same adenoviral vector. To obtain tissue-specific expression of the target gene, we coupled the regulator to a liver-specific promoter. Infection of HepG2 cells and experimental mice with the adenovirus resulted in consistently high induction levels of hGH in the presence of RU486, while the transgene expression was undetectable in the absence of the ligand. Taken together, our regulatable adenoviral vector represents an important tool for transgene regulation that can be used for potentially diverse applications, ranging from tissue-specific gene expression in transgenic animals to human gene therapy.

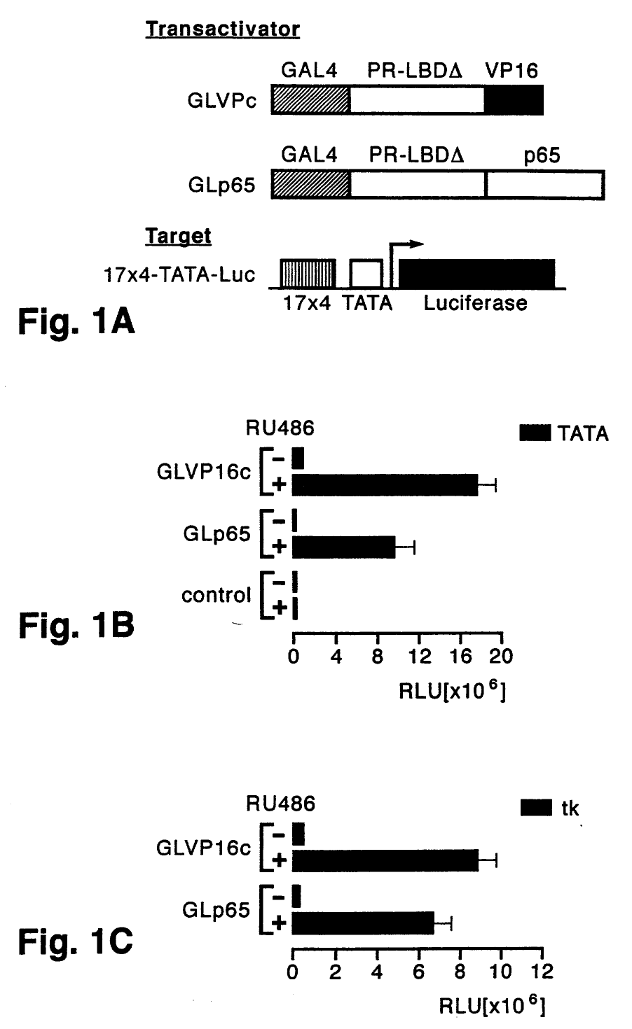

The ability to transfer foreign genes into an organism is a major goal in a wide variety of applications ranging from tissue-cultured cells to transgenic animals and human gene therapy. Since endogenous genes are expressed at specific time points and at specific levels, constitutive expression of transferred genes is generally unsatisfactory. Different regulatory systems have been developed to approach this problem of target gene regulation. We have recently developed a novel regulatable system (Wang, Y., et al., Proc. Nat'l. Acad. Sci. USA, 91:8180-4, 1994; Wang, Y., et al., Gene Therapy, 4:432-41, 1997; Wang, Y., et al., Nature Biotechnology, 15:239-43, 1997) which can be used to express a target gene in vivo in a specific tissue, at a desired time and under the control of an oral, nontoxic chemical. This system utilized a chimeric regulator, GLVP, consisting of a modified human progesterone receptor ligand binding domain (PRLBDΔ) fused to the yeast GAL4 DNA binding domain (DBD) and the HSV, herpes simplex virus protein activation domain, VP16 transcriptional activation domain (FIG. 1A).

In the presence of the anti-progestin mifepristone (RU486), but not endogenous molecules present in mammalian tissues and organs, this chimeric regulator binds to a target gene containing the 17-mer GAL4 upstream activation sequence (UAS) and results in efficient ligand-inducible transactivation of the target gene (Wang, Y., et al., Gene Therapy, 4:432-41, 1997; Wang, Y., et al., Nature Biotechnology, 15:239-43, 1997). The gene regulator responded to RU486 at a concentration that has no endogenous anti-progesterone or anti-glucocorticold activity.

By replacing VP16 with a variety of human-derived activation domains, we show that a region of the human p65 (Schmitz, M. L. & Baeuerle, P. A., EMBO Journal, 10:3805-17, 1991), a member of the NF-κB family, allows retention of the potent inducibility of GLVP and precludes a possible immune response caused by the anticipated antigenicity of the VP16 domain.

In order to facilitate initial delivery of our inducible system in vitro and in vivo, we have developed an adenoviral vector-mediated gene transfer strategy. Previous results have shown that viral delivery generally has the following inherent limitations: (i) expression of viral proteins in infected cells is believed to trigger a cellular immune response that precludes long-term expression of the transferred gene-; and (ii) the insert capacity of adenoviral vectors has been previously limited to 8 kb of transgenic sequence. However, an adenoviral vector has been recently constructed (Kochanek, S., et al., Proc Natl Acad Sci USA, 93:5731-6, 1996; Schiedner, G., et al., Nat Genet, 18:180-3, 1998), which contains no viral coding sequences and possesses a very large insert capacity (up to 35 kb).

To combine this improved adenoviral vector with our regulatory system we have inserted into the vector a single regulatory cassette containing the regulator GLp65 and a regulatable human growth hormone (hGH) target gene coupled to the 17mer GAL4 binding site. To obtain tissue-specific expression of the target gene, we have coupled the regulator to the liver-specific transthyretin (TTR) promoter region (Yan, C., et al., Embo J, 9:869-78, 1990; Wu, H., et al., Wade, M., et al., Genes Dev, 10:245-60, 1996). Finally, to investigate the effect of an insulator on the regulatable adenoviral target gene expression, the 5′ element of the chicken β-globin domain (Chung, J. H., et al., Cell, 74:505-14, 1993) has been inserted between the target gene and the regulator. Using this adenoviral vector in combination with our inducible regulator system, we successfully demonstrate potent inducible expression of hGH in both cultured (liver tumor-derived) HepG2 cells and in experimental mice.

Recently, a variety of regulatory systems have been developed with the goal of regulating target gene expression (Shockett, P. E. & Schatz, D. G., Proc. Nat'l. Acad. Sci. USA, 93:5173-6, 1996; Gossen, M, et al., Trends in Biochemical Sciences, 18:471-5, 1993; Baim, S. B., et al., Proc. Nat'l. Acad. Sci. USA, 88:5072-6, 1991; Gossen, M., et al., Science, 268:1766-9, 1995; No, D., et al., Proc. Nat'l. Acad. Sci. USA, 93:3346-51, 1996). The desirable goals of such inducible systems are to achieve low basal expression with a high inducibility and rapid kinetics of induction upon administration of a non-toxic and easily deliverable drug.

The combination of our regulatory system with a high capacity adenoviral vector as described here made certain regulator modifications desirable. To facilitate future applications of our regulatory system in human gene therapy, it was desirable to replace the viral VP16 activation domain with other mammalian transcription factor activation domains because there is a higher probability that the VP16 protein could cause an immune response in humans. In addition, high expression levels of the VP16 activation domain are known to have a squelching effect and can be toxic to cells (Ptashne, M., et al., Nature, 346:329-31, 1990 and Triezenberg, S. J., et al., Genes Dev, 2:718-29, 1988).

After replacing VP16 with a variety of human derived activation domains we chose p65, a partner of NF-κB in the human RelA heterodimeric transcription factor, because it is known to possess a strong potential to activate transcription (Schmitz, M. L. & Baeuerle, P. A., EMBO Journal, 10:3805-17, 1991). In comparison, the VP16 and the p65 derived regulators show similar inducibility upon RU486 induction. In fact, the magnitude of the GLp65 induction is superior to that of the GLVP regulator due to the low basal activity of the GLp65 regulator. Because of its non-viral p65 activation domain and its strong inducibility the modified version (GLp65) of our inducible regulator has potential for use in human gene therapy.

To complement the modification of our regulatory system and to enhance the efficiency of in vivo delivery we decided to use a high-capacity adenoviral vector lacking all viral sequences (Kochanek, S., et al., Proc Natl Acad Sci USA, 93, 5731-6, 1996; Schiedner, G., et al., Nat Genet, 18, 180-3, 1998; and Parks, R. J., et al., Proc. Nat'l. Acad. Sci. USA, 93, 13565-70, 1996) which could minimize toxicity and immunogenicity of the viral proteins known to cause short duration of target gene expression. Infecting mice with the regulatable adenoviral vector we show RU486 dependent induction of the transgene. The initial time delay of 8 days between viral infection and hGH inducibility upon RU486 administration was somewhat unexpected, since other investigations using the new adenoviral vector have shown that when under control of a constitutive promoter, target gene expression can be detected 3 days post-infection (Schiedner, G., et al., Nat Genet, 18, 180-3, 1998).

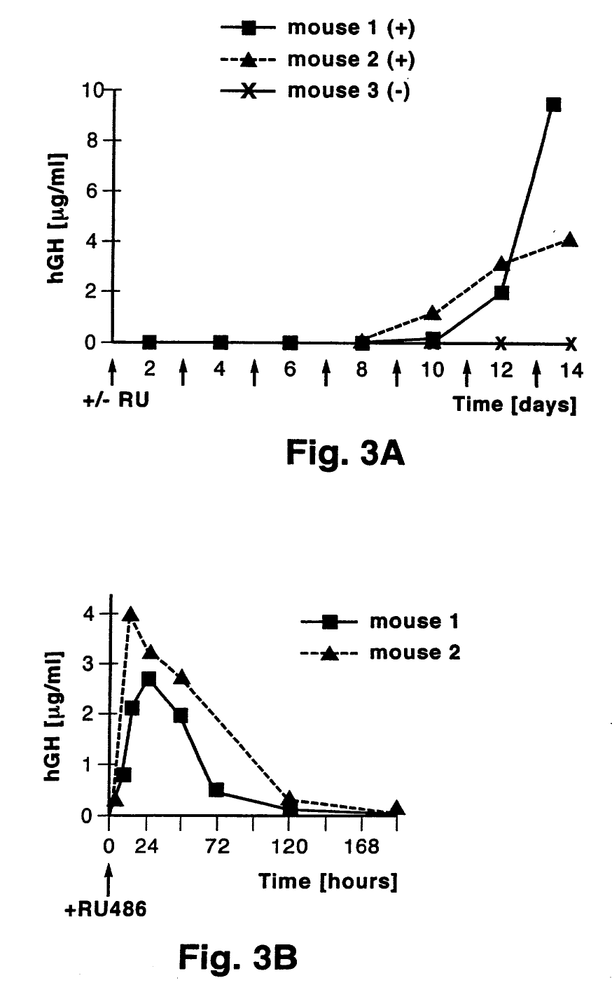

Two alternative reasons could explain the difference: (i) the liver-specific promoter used in our investigations to drive the GLp65 expression might need a defined concentration of transcription factors and the assembly of specific transcriptional complexes might take some time, both of which could contribute to the delay of the regulator expression; or (ii) to be able to induce target gene expression in a potent manner, the regulator concentrations need to exceed a specific threshold which slowly builds up in the cells within the first few days after viral infection (FIG. 3A).

Once the transduced gene is inducible in the animals, our regulatory system shows a fast response to the inducer such that maximal transgene expression can be attained 12 hours after induction (FIG. 3B).

We observed different expression levels of the transgene upon administration of different amounts of RU486. This is an important goal, since for gene therapy the expression level of most transgenes appears to require a therapeutic “window” in which a successful gene transfer may be accomplished. The doses of RU486 needed for induction in our regulatory system (0.1-0.5 mg/kg) is far below levels where RU486 is used as an antiprogestin (10 mg/kg) together with prostaglandin to terminate pregnancy. Administration of RU486 at levels much higher than those necessary for transgene induction in our regulatory system have been safely administered to patients on a daily basis to treat different diseases (Grunberg, S. M., et al., J Neurosurg, 74, 861-6, 1991 and Brogden, R. N., et al., Drugs, 45, 384-409, 1993). Thus it is likely RU486, at this low concentration can serve as a potent inducer for human gene therapy, even for a prolonged period of time.

Chromatin insulators are involved in position independent expression of transgenes and have been shown to confer chromosomal integration site-independent transgene expression in transgenic mice (Wang, Y., et al., Nature Biotechnology, 15:239-43, 1997). Using this insulator in combination with our regulatable adenoviral vector we obtained different effects, depending on whether the infection was carved out in transient transfection or in vivo.

The ability to transfer large DNA elements and the ability to regulate the transgene expression over a long period of time are important criteria for the success of human gene therapy. Here we combine a high capacity adenoviral vector deficient of all viral coding sequences with a single regulatory expression cassette to achieve persistent and inducible transgene expression in vivo. Induction was comparable when RU486 was given by I.P. or oral routes. This combination represents an important advancement for transgene regulation that can be used for diverse applications, ranging from tissue-specific gene expression in transgenic animals to chronic human gene therapy.

Cell Transformation

One embodiment of the present invention includes cells transformed with nucleic acid encoding for the modified receptor. Once the cells are transformed, the cells will express the protein, polypeptide, or RNA encoded for by the nucleic acid. Cells include but are not limited to joints, lungs, muscle and skin. This is not intended to be limiting in any manner.

The nucleic acid which contains the genetic material of interest is positionally and sequentially oriented within the host or vectors such that the nucleic acid can be transcribed into RNA and, when necessary, be translated into proteins or polypeptides in the transformed cells. A variety of modified proteins and polypeptides can be expressed by the sequence in the nucleic acid cassette in the transformed cells.

Transformation can be done either by in vivo or ex vivo techniques. One skilled in the art will be familiar with such techniques for transformation. Transformation by ex vivo techniques includes co-transfecting the cells with DNA containing a selectable marker. This selectable marker is used to select those cells which have become transformed. Selectable markers are well known to those who are skilled in the art.

For example, one approach to gene therapy for muscle diseases is to remove myoblasts from an affected individual, genetically alter them in vitro, and reimplant them into a receptive locus. The ex vivo approach includes the steps of harvesting myoblasts cultivating the myoblasts, transducing or transfecting the myoblasts, and introducing the transfected myoblasts into the affected individual.

The myoblasts may be obtained in a variety of ways. They may be taken from the individual who is to be later injected with the myoblasts that have been transformed or they can be collected from other sources, transformed and then injected into the individual of interest.

Once the ex vivo myoblasts are collected, they may be transformed by contacting the myoblasts with media containing the nucleic acid transporter and maintaining the cultured myoblasts in the media for sufficient time and under conditions appropriate for uptake and transformation of the myoblasts. The myoblasts may then be introduced into an appropriate location by injection of cell suspensions into tissues. One skilled in the art will recognize that the cell suspension may contain: salts, buffers or nutrients to maintain viability of the cells; proteins to ensure cell stability; and factors to promote angiogenesis and growth of the implanted cells.

In an alternative method, harvested myoblasts may be grown ex vivo on a matrix consisting of plastics, fibers or gelatinous materials which may be surgically implanted in an appropriate location after transduction. This matrix may be impregnated with factors to promote angiogenesis and growth of the implanted cells. Cells can then be reimplanted.

Administration

Administration as used herein refers to the route of introduction of a vector or carrier of DNA into the body. Administration may include intravenous, intramuscular, topical, or oral methods of delivery. Administration can be directly to a target tissue or through systemic delivery.

In particular, the present invention can be used for treating disease or for administering the formulated DNA expression vectors capable of expressing any specific nucleic acid sequence. Administration can also include administering a regulatable vector discussed above. Such administration of a vector can be used to treat disease. The preferred embodiment is by direct injection to the target tissue or systemic administration.

A second step is the delivery of the DNA vector to the nucleus of the target cell where it can express a gene product. In the present invention this is accomplished by an adenoviral vector. The amount of expression vector delivered into the cells may be controlled by titration of the adenoviral vector.

The delivery and formulation of any selected vector construct will depend on the particular use for the expression vectors. In general, a specific formulation for each vector construct used will focus on vector uptake with regard to the particular targeted tissue, followed by demonstration of efficacy. Uptake studies will include uptake assays to evaluate cellular uptake of the vectors and expression of the tissue specific DNA of choice. Such assays will also determine the localization of the target DNA after uptake, and establish the requirements for maintenance of steady-state concentrations of expressed protein. Efficacy and cytotoxicity can then be tested. Toxicity will not only include cell viability but also cell function.

DNA uptake by cells associated with fluid spaces have the unique ability to take up DNA from the extracellular space after simple injection of purified DNA preparations into the fluid spaces. Expression of DNA by this method can be sustained for several months.

Incorporating DNA by formulation into particulate complexes of nanometer size that undergo endocytosis increases the range of cell types that will take up foreign genes from the extracellular space.

Formulation can also involve DNA transporters which are capable of forming a non-covalent complex with DNA and directing the transport of the DNA through the cell membrane. This may involve the sequence of steps including endocytosis and enhanced endosomal release. It is preferable that the transporter also transport the DNA through the nuclear membrane. See, e.g., the following applications all of which (including drawings) are hereby incorporated by reference herein: (1) Woo et al., U.S. Ser. No. 07/855,389, entitled “A DNA Transporter System and Method of Use” filed Mar. 20, 1992; (2) Woo et al., PCT/US93/02725, entitled “A DNA Transporter System and Method of Use”, (designating the U.S. and other countries) filed Mar. 19, 1993; and (3) continuation-in-part application by Woo et al., entitled “Nucleic Acid Transporter Systems and Methods of Use”, filed Dec. 14, 1993, assigned U.S. Ser. No. 08/167,641.

In addition, delivery can be cell specific or tissue specific by including cell or tissue specific promoters. Furthermore, mRNA stabilizing sequences (3′ UTR's) can be used to provide stabilized modified receptor molecules. Such stabilizing sequences increase the half-life of mRNAs and can be cell or tissue specific. The above is discussed in more detail in U.S. Pat. No. 5,298,422 (Schwartz et al.) and U.S. application Ser. No. 08/209,846 (Schwartz et al.), filed Mar. 9, 1994, entitled “Expression Vector Systems and Method of Use.” Both of these, the whole of which, are incorporated by reference herein, including drawings.

In a preferred method of administration involving a DNA transporter system, the DNA transporter system has a DNA binding complex with a binding molecule capable of non-covalently binding to DNA which is covalently linked to a surface ligand. The surface ligand is capable of binding to a cell surface receptor and stimulating entry into the cell by endocytosis, pinocytosis, or potocytosis. In addition, a second DNA binding complex is capable of non-covalently binding to DNA and is covalently linked to a nuclear ligand. The nuclear ligand is capable of recognizing and transporting a transporter system through a nuclear membrane. Additionally, a third DNA binding complex may be used which is also capable of non-covalently binding to DNA. The third binding molecule is covalently linked to an element that induces endosomal lysis or enhanced release of the complex from the endosome after endocytosis. The binding molecules can be spermine, spermine derivatives, histones, cationic peptides and/or polylysine. See also Szoka, C. F., Jr. et al., Bioconjug. Chem. 4:85-93 (1993); Szoka, F. C., Jr. et al., P.N.A.S., 90:893-897 (1993).

Transfer of genes directly has been very effective. Experiments show that administration by direct injection of DNA into joint tissue results in expression of the gene in the area of injection. Injection of plasmids containing the modified receptors into the spaces of the joints results in expression of the gene for prolonged periods of time. The injected DNA appears to persist in an unintegrated extrachromosomal state. Thus, direct objection of the viral vector is a preferred embodiment.

The formulation used for delivery may also be by liposomes or cationic lipids. Liposomes are hollow spherical vesicles composed of lipids arranged in a similar fashion as those lipids which make up the cell membrane. They have an internal aqueous space for entrapping water soluble compounds and range in size from 0.05 to several microns in diameter. Several studies have shown that liposomes can deliver nucleic acids to cells and that the nucleic acid remains biologically active. Cationic lipid formulations such as formulations incorporating DOTMA has been shown to deliver DNA expression vectors to cells yielding production of the corresponding protein. Lipid formulations may be non-toxic and biodegradable in composition. They display long circulation half-lives and recognition molecules can be readily attached to their surface for targeting to tissues. Finally, cost effective manufacture of liposome-based pharmaceuticals, either in a liquid suspension or lyophilized product, has demonstrated the viability of this technology as an acceptable drug delivery system. See Szoka, F. C., Jr. et al., Pharm. Res., 7:824-834 (1990); Szoka, F. C., Jr. et al., Pharm. Res., 9:1235-1242 (1992).

The chosen method of delivery should result in nuclear or cytoplasmic accumulation and optimal dosing. The dosage will depend upon the disease and the route of administration but should be between 1-1000 μg/kg of body weight. This level is readily determinable by standard methods. It could be more or less depending on the optimal dosing. The duration of treatment will extend through the course of the disease symptoms, possibly continuously. The number of doses will depend upon disease, the formulation and efficacy data from clinical trials.

With respect to vectors, the pharmacological dose of a vector and the level of gene expression in the appropriate cell type includes but is not limited to sufficient protein or RNA to either: (1) increase the level of protein production; (2) decrease or stop the production of a protein; (3) inhibit the action of a protein; (4) inhibit proliferation or accumulation of specific cell types; and (5) induce proliferation or accumulation of specific cell types. As an example, if a protein is being produced which causes the accumulation of inflammatory cells within the joint, the expression of this protein can be inhibited, or the action of this protein can be interfered with, altered, or changed.

Using Episomal Vectors for Persistent Expression

In each of the foregoing examples, transient expression of recombinant genes induces the desired biological response. In some diseases more persistent expression of recombinant genes is desirable. This is achieved by adding elements which enable extrachromosomal (episomal) replication of DNA to the structure of the vector. Vectors capable of episomal replication are maintained as extrachromosomal molecules and can replicate. These sequences will not be eliminated by simple degradation but will continue to be copied. Episomal vectors provide prolonged or persistent, though not necessarily stable or permanent, expression of recombinant genes in the joint. Persistent as opposed to stable expression is desirable to enable adjustments in the pharmacological dose of the recombinant gene product as the disease evolves over time.

Formulations for Gene Delivery into Cells of the Joint

Initial experiments used DNA in formulations for gene transfer into cells of the joint. This DNA is taken up by synovial cells during the process of these cells continually resorbing and remodeling the synovial fluid by secretion and pinocytosis. Gene delivery is enhanced by packaging DNA into particles using cationic lipids, hydrophilic (cationic) polymers, or DNA vectors condensed with polycations which enhance the entry of DNA vectors into contacted cells. Formulations may further enhance entry of DNA vectors into the body of the cell by incorporating elements capable of enhancing endosomal release such as certain surface proteins from adenovirus, influenza virus hemagglutinin, synthetic GAL4 peptide, or bacterial toxins. Formulations may further enhance entry of DNA vectors into the cell by incorporating elements capable of binding to receptors on the surface of cells in the joint and enhancing uptake and expression. Alternatively, particulate DNA complexed with polycations can be efficient substrates for phagocytosis by monocytes or other inflammatory cells. Furthermore, particles containing DNA vectors which are capable of extravasating into the inflamed joint can be used for gene transfer into the cells of the joint. One skilled in the art will recognize that the above formulations can also be used with other tissues as well.

Induction of “Steroid Response” by Gene Transfer of Steroid Receptors into Cells of the Joint

Current therapy for severe arthritis involves the administration of pharmacological agents including steroids to depress the inflammatory response. Steroids can be administered systemically or locally by direct injection into the joint space.

Steroids normally function by binding to receptors within the cytoplasm of cells. Formation of the steroid-receptor complex changes the structure of the receptor so that it becomes capable of translocating to the nucleus and binding to specific sequences within the genome of the cell and altering the expression of specific genes. Genetic modifications of the steroid receptor can be made which enable this receptor to bind non-natural steroids. Other modifications can be made to create a modified steroid receptor which is “constitutively active” meaning that it is capable of binding to DNA and regulating gene expression in the absence of steroid in the same way that the natural steroid receptor regulates gene expression after treatment with natural or synthetic steroids.

Of particular importance is the effect of glucocorticoid steroids such as cortisone, hydrocortisone, prednisone, or dexamethasone which are effective drugs available for the treatment of arthritis. One approach to treating arthritis is to introduce a vector in which the nucleic acid cassette expresses a genetically modified steroid receptor into cells of the joint, e.g., a genetically modified steroid receptor which mimics the effect of glucocorticoid but does not require the presence of glucocorticoid for effect. This is achieved by expression of a fusion receptor protein discussed above or other modified glucocorticoid receptors such as ones which are constitutively active within cells of the joint. This induces the therapeutic effects of steroids without the systemic toxicity of these drugs.

Alternatively, construction of a steroid receptor which is activated by a novel, normally-inert steroid enables the use of drugs which would affect only cells taking up this receptor. These strategies obtain a therapeutic effect from steroids on arthritis without the profound systemic complications associated with these drugs. Of particular importance is the ability to target these genes differentially to specific cell types (for example synovial cells versus lymphocytes) to affect the activity of these cells.

The steroid receptor family of gene regulatory proteins is an ideal set of such molecules. These proteins are ligand activated transcription factors whose ligands can range from steroids to retinoic acid, fatty acids, vitamins, thyroid hormones and other presently unidentified small molecules. These compounds bind to receptors and either activate or repress transcription.

A preferred receptor of the present invention is modification of the glucocorticoid receptor, i.e., the fusion protein receptor. These receptors can be modified to allow them to bind various ligands whose structure differs from naturally occurring ligands. For example, small C-terminal alterations in amino acid sequence, including truncation, result in altered affinity of ligand binding to the progesterone receptor. By screening receptor mutants, receptors can be customized to respond to ligands which do not activate the host cell endogenous receptors.

A person having ordinary skill in the art will recognize, however, that various mutations, for example, a shorter deletion of carboxy terminal amino acids, will be necessary to create useful mutants of certain steroid hormone receptor proteins. Steroid hormone receptors which may be modified are any of those receptors which comprise the steroid hormone receptor superfamily, such as receptors including the estrogen, progesterone, glucocorticoid-α, glucocorticoid-β, mineral corticoid, androgen, thyroid hormone, retinoic acid, and Vitamin D3 receptors.

Direct DNA Delivery to Muscle

Diseases that result in abnormal muscle development, due to many different reasons can be treated using the above modified glucocorticoid receptors. These diseases can be treated by using the direct delivery of genes encoding for the modified glucocorticoid receptor of the present invention resulting in the production of modified receptor gene product. Genes which can be repressed or activated have been outlined in detail above.

Direct DNA Delivery to the Lungs

Current therapy for severe asthma involves the administration of pharmacological agents including steroids to inhibit the asthma response. Steroids can be administered systemically or locally by direct instillation or delivery into the lungs.

Of particular importance is the effect of glucocorticoid steroids such as cortisone, hydrocortisone, prednisone, or dexamethasone which are the most important-effective drugs available for the treatment of asthma. One approach to treating asthma is to introduce a vector in which the nucleic acid cassette expresses a genetically modified steroid receptor into cells of the lungs, e.g., a genetically modified steroid receptor which mimics the effect of glucocorticoid but does not require the presence of glucocorticoid for effect. This is achieved by expression of the fusion proteins discussed above or other modified glucocorticoid receptors such as ones which are constitutively active within cells of the lungs. This induces the therapeutic effects of steroids without the systemic toxicity of these drugs.

Alternatively, construction of a steroid receptor which is activated by a novel, normally-inert steroid enables the use of drugs which would affect only cells taking up this receptor. These strategies obtain a therapeutic effect from steroids on asthma without the profound systemic complications associated with these drugs. Of particular importance is the ability to target these genes differentially to specific cell types (for example alveoli of the lungs) to affect the activity of these cells.

The steroid receptor family of gene regulatory proteins is an ideal set of such molecules. These proteins are ligand-activated transcription factors whose ligands can range from steroids to retinoids, fatty acids, vitamins, thyroid hormones, and other presently unidentified small molecules. These compounds bind to receptors and either up-regulate or down-regulate transcription.

The preferred receptor of the present invention is the modified glucocorticoid receptor. These receptors can be modified to allow them to bind various ligands whose structure differs from naturally occurring ligands. For example, small C-terminal alterations in amino acid sequence, including truncation, result in altered affinity of the ligand and altered function. By screening receptor mutants, receptors can be customized to respond to ligands which do not activate the host cells own receptors.

A person having ordinary skill in the art will recognize, however, that various mutations, for example, a shorter deletion of carboxy terminal amino acids, will be necessary to create useful mutants of certain steroid hormone receptor proteins. Steroid hormone receptors which may be modified are any of those receptors which comprise the steroid hormone receptor superfamily, such as receptors including the estrogen, progesterone, glucocorticoid-β, glucocorticoid-α, mineral corticoid, androgen, thyroid hormone, retinoic acid, and Vitamin D3 receptors.

### EXAMPLES

While the present invention is disclosed by reference to the details for the following examples, it is to be understood that this disclosure is intended in an illustrative rather than limiting sense, as it is contemplated that modifications will readily occur to those skilled in the art, within the spirit of the invention and the scope of the appended claims.

Materials and Methods

Construction of GLp65. A HindIII-BamHl fragment of 680 bp was isolated from PAP cytomegalovirus (hereinafter referred to as “CMV”) CMV-GL914VPc′SV (Wang, Y., et al., Nature Biotechnology, 15:239-43, 1997 and cloned into the HindIII-BamHI site of a pUC18 plasmid. The resulting construct was named pUC-LBD914VPc′SV. The p65 activation domain (residues 286-550) was isolated from Gal4-p65 long (Schmitz, M. L. & Baeuerle, P. A., EMBO Journal, 10:3805-17, 1991), by an EcoRl-BamHI digest. This fragment was ligated with a Sal I linker TCGACGAGATATCAAGCAG to pUC-LBD914VPc′SV after VP16 was excised by Sal I-BamHI and the resulting plasmid was named pUC-LBD914p65. After digesting both, this construct and PAP CMV-GL914VPc′SV with HindIII-BamHI, the resulting fragments were ligated together to create the new chimeric regulator GLp65.

Construction of vector containing both regulator and target gene. Reporter plasmid p17x4-TATA-Luc (Luc, luciferase) containing the adenovirus major late Elb TATA box and p17x4-tk-Luc containing the thymidine kinase gene promoter have been described (Smith, C. L., et al., Proc Natl Acad Sci USA, 93:8884-8, 1996). To combine our regulator GLp65 with an hGH target gene on one plasmid, we created the following constructs. GLp65 was first isolated from PAP CMV-GLp65 by a complete Kpnl and a partial BamHI digestion to generate a BamHI-KpnI fragment. This fragment was then ligated to PAP TTRBSV (Wang, Y., et al., Nature Biotechnology, 15:239-43, 1997) to create PAP TTRB-GLp65SV.

Secondly, TTRB GLVP SV was excised from PAP TAGH TTRB GLVP SV (Wang, Y., et al., Nature Biotechnology, 15:239-43, 1997) by AscI-PacI digestion. TTRB-GLp65SV was then inserted in the Ascl-Pacl digested vector, resulting in PAP-GH-GLp65 which consists of the human growth hormone genomic gene under the control of a TATA promoter and the GLp65 regulator driven by the liver-specific promoter TTRB (Yan, C., et al., Embo J, 9:869-78, 1990; and Wu, H., et al., Genes Dev, 10:245-60, 1996).

PAP-GH-H-GLp65 was constructed in a similar manner, except that an additional insulator sequence from the 5′ element of the chicken β-globin domain (Yan, C., et al., Embo J, 9:869-78, 1990) was inserted by digesting PAP-GH-GLp65 with Ascl, -GH-GLp65 blunt-ended and ligated with a blunt-ended 2.4 kb BamHI-XhoI fragment from pBS-HS4.

Adenoviral constructs. The plasmid pSTK119, which was used to construct the adenoviral vectors, has a 22.5-kb insert in the multiple cloning site of pBluescript KSII with, from the left to the right, the following features: the left terminus of adenovirus type 5 (nt 1-440), a 16054 bp EclXI/PmeI fragment of the human HPRT gene (nt 1799-17853 in gb: humhprtb), a 6545 bp EcoRV fragment of the C346 cosmid (nt 10205-16750 in gb: L31948) and the right terminus of adenovirus type 5 (nt 35818-35935). To construct regulatable adenoviral vectors the regulatory expression cassette was isolated from PAP-GH-GLp65 by NotI digestion and subcloned into the EclXI site of AdSTK119, resulting in GH-GLp65. The adenoviral vector GH-H-GLp65 was constructed in an analogous manner using an insert isolated from PAP-GH-H-GLp65.

Cell Culture and Transient Transfection Assays. HeLa (human epithelial cervix carcinoma) cells were grown in DMEM supplemented with 5% fetal bovine serum. Twenty-four hours before transfection, 3×10⁵ cells were plated on 6 well collagen-coated dishes in DMEM with 5% dextran-coated charcoal stripped serum. Cells were transfected with the indicated amounts of DNA using Lipofectin (Life Technologies) according to the manufacturer's protocol. 18 hours later, cells were washed with 1×HBSS and DMEM, plus 5% stripped serum before an indicated amount of RU486 (dissolved in 80% ethanol) was added. After 36 hours, the cells were harvested and cell extract was assayed for luciferase activity using the luciferase assay system (Promega). Data is presented as the mean (ISD) of triplicate values.

Rescue of GH-GLp65 and GH-H-GLp65 adenoviral vectors. Adenoviral constructs were cleaved by Pmel and transfected into 293-Cre4 cells. Subsequently, the cells were infected with loxP helper virus AdLC8cluc (Parks, R. J., et al., Proc Natl Acad Sci USA, 93:13565-70, 1996.). To increase the titer, vector lysates were passed through 293-Cre4 cells several times and remaining helper virus were separated by CsCl equilibrium density centrifugation. The detailed procedure for adenoviral rescue and virus characterization has been previously described (Schiedner, G., et al., Nat Genet, 18:180-3, 1998, which is incorporated herein by reference in its entirety, including any drawings). The concentration of the viral particles for GH-H-GLp65 was 4.1×10¹¹/ml and for GH-GLp65 was 7.8×10¹¹/ml. The particle/infectious units' ratio was 20:1 with both vectors. The contamination of lox-P helper virus in the virus preparation was about 0.01-0.05%. In addition, the viral preparation did not contain any replication competent adenoviruses (RCA).

Infection of HepG2 cells. HepG2 (human epithelial hepatoblastoma) cells were maintained as described. 2×10⁵ cells were plated onto 6 well dishes in DMEM with 5% dextran-coated charcoal-stripped serum. 2×10⁵ cells were infected with 1×10⁹ viral particles (5×10⁷) infectious units, at a multiplicity of infection (MIO) of 250. The viral particles were left on the cells for 3 hours, then cells were washed with 1×HBSS and DMEM containing 5% stripped serum and the indicated amount of RU486 was added. The levels of hGH in the medium were measured 24 hours later using a radioimmunoassay (Nichols Institute Diagnostics) according to the manufacturer's protocol.

Mouse strains. C57B46 mice were purchased from the Jackson Laboratory. All mice were 8-10 weeks old at the time of injection.

hGH analysis in adenoviral infected mice. C57BL/6 mice were infected by tail vein injection with 2×10⁹ infectious units of either GH-GLp65 or GH-H-GLp65 diluted in phosphate-buffer saline (PBS). Mice were given RU486 (dissolved in sesame oil) or vehicle control at the specified dose and at indicated time points by intraperitoneal or oral routes. At definite time points mice were bled from the ophthalmic orbit using a glass capillary or from the tail vein. Serum was obtained by blood incubation for 1 h at room temperature followed by centrifugation of the samples for 10 minutes at 10,000 rpm. Serum hGH levels were detected using a radio-immunoassay. When hGH levels exceeded the assay limit of 50 ng/ml, serum dilutions were performed in 1×PBS.

### Example 1

### Regulator Modifications

We replaced the viral VP16 activation domain with the human p65 activation domain of GLVPc′ (residues 286-550) and constructed the regulator GLp65 (FIG. 1A). To compare the ability of these regulators to induce a target gene in an RU486 dependent manner, we cotransfected regulator together with a reporter plasmid containing the luciferase gene driven by four copies of the consensus GAL4-binding site (17-mer) upstream of either a TATA or tk promoter into HeLa cells.

FIGS. 1B and 1C, show the potential of our different regulators to activate target gene expression in transient transfection. Using a TATA promoter, the basal activity of GLp65 is significantly lower than that of GLVPc′ in the absence of RU486. When RU486 was added both regulators showed ligand dependent target gene expression. GLVPc′ induced slightly higher expression levels of the target gene as compared to GLp65. Since basal expression of GLp65 is usually lower, induction with this construct results in higher fold activation. Transfecting the expression plasmid backbone as a control resulted in no activation of the reporter plasmid. When using a tk promoter linked to the reporter plasmid (FIG. 1C), both regulators show similar low basal expression in the absence of RU486, as well as similar inducible target gene expression upon RU486 administration.

These results demonstrate that the new regulator GLp65 which contains the human p65 activation domain, has a similar potency in the induction of target gene expression in transient transfection when compared to GLVPc′. In addition, a lower basal expression level was observed in the absence of the ligand. Overall, the performance of our regulatory system seems to be promoter dependent. In the case of the GLp65 regulator, optimal RU486 dependent transgene regulation appears to require a TATA promoter.

### Example 2

### Construction of a Regulatable Adenoviral Vector

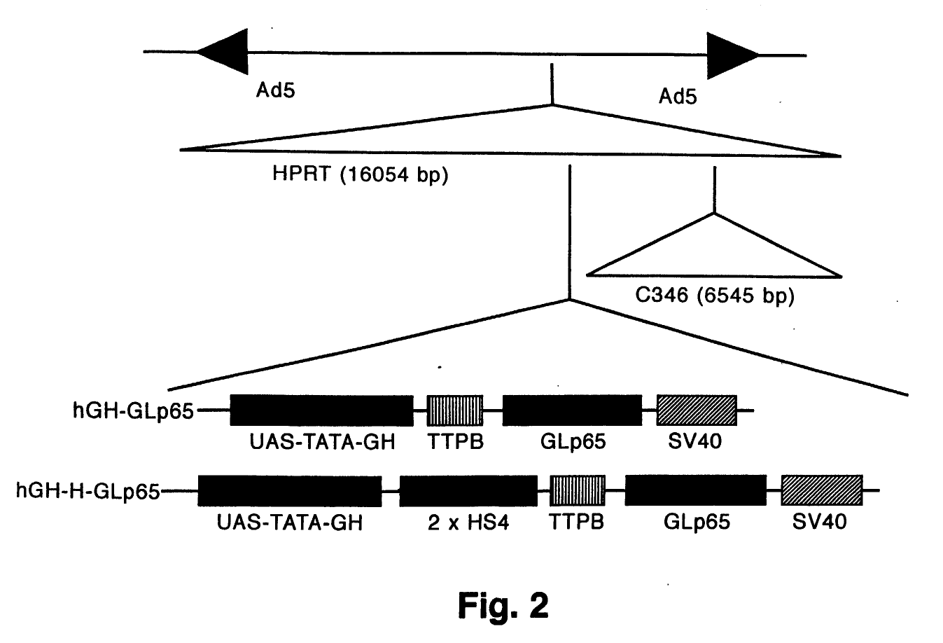

In order to facilitate initial delivery of our inducible system in vivo, we proposed the use of an adenoviral vector-mediated gene transfer strategy where the virus has all viral coding sequences removed. Into this vector we inserted a regulatable expression cassette (FIG. 2). To achieve tissue-specific expression of the regulator and target protein, we first placed the regulator GLp65 encoding a GAL4 DNA-binding-site, PR-ligand binding domain and a p65 activation domain, together with the SV40 polyA under control of the TTRB fragment, which contains a liver-specific promoter and enhancer.

To combine the regulator with the target gene, we fused the coding sequence for hGH, under control of a GAL4 binding site and a TATA promoter, together with the GLp65 transcription unit. This adenoviral construct was named hGH-GLp65. We used a 5′ element of the chicken beta-globin domain (2×HS4) to investigate the insulator effect on adenoviral mediated gene transfer. To do this, we created a second adenoviral construct (hGH-H-GLp65) where we inserted a chromosomal insulator between the hGH and the GLp65 cassette.

### Example 3

### Inducible hGH Expression Using Adenoviral Constructs in Transient Transfection Assays

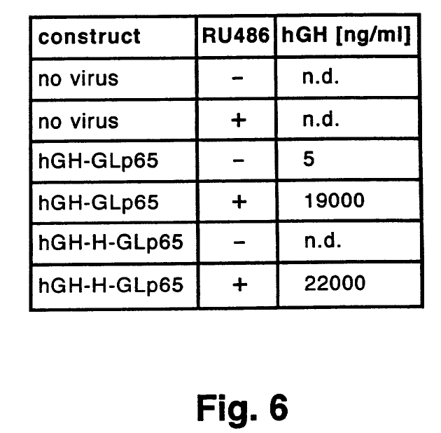

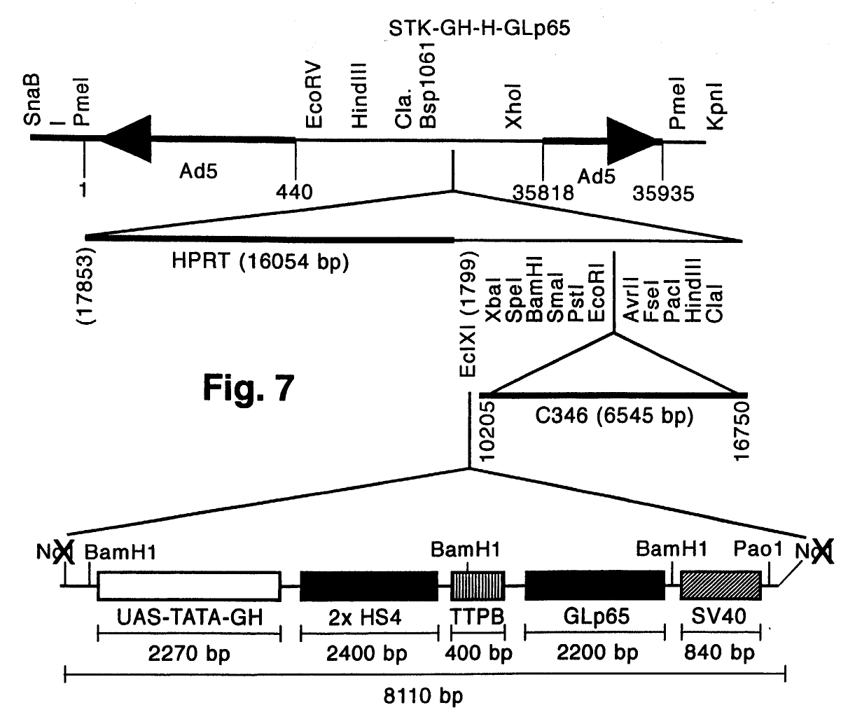

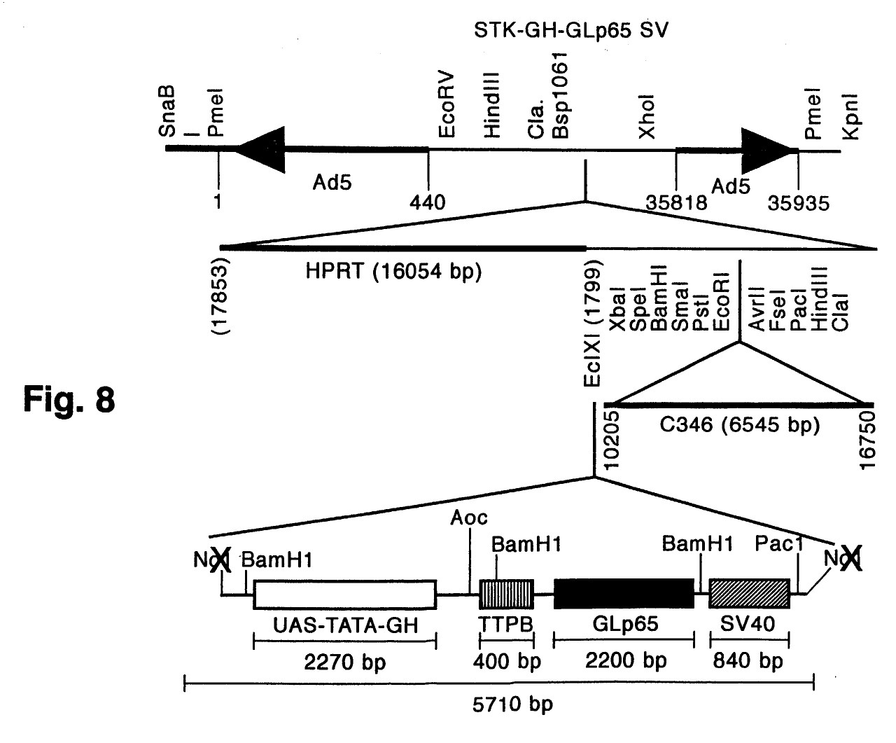

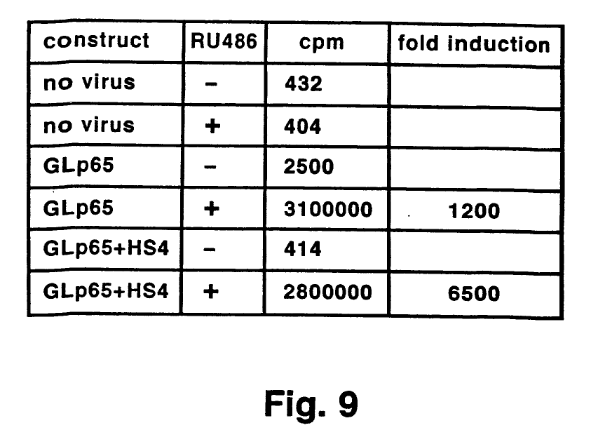

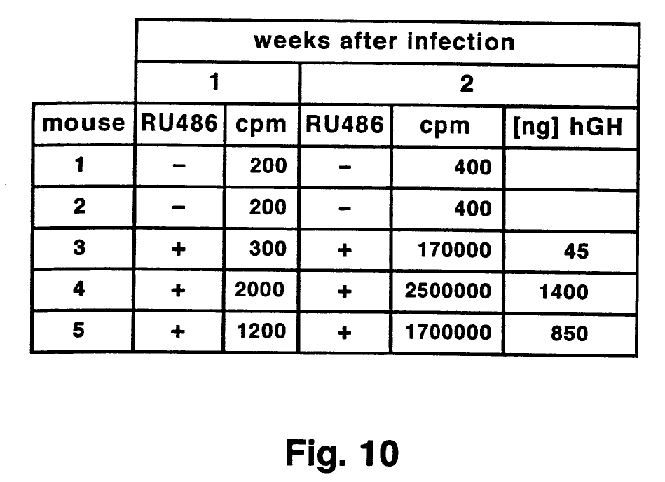

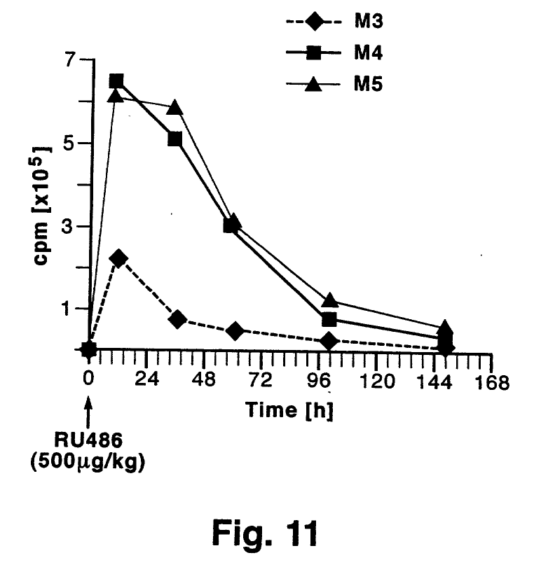

After generating the adenoviral particles, we examined the ability of the viral vector to infect hepatocytes and regulate expression of hGH in cell culture. The infection was carried out for three hours, then the medium was changed and RU486 added as appropriate hGH was measured in the medium after 48 hours by a radio-immunoassay. As seen in FIG. 6, both adenoviral vectors regulate the expression of hGH in an RU486-dependent manner and express in the presence of RU486 up to 20 μg hGH per ml medium. In the absence of RU486, hGH-H-GLp65 harboring the insulator shows no detectable expression of hGH whereas hGH-GLp65 seems to express hGH at a very low level in the absence of the ligand.

### Example 4

### Inducible HGH Expression Using Adenoviral Constructs in vivo

In order to assess the ability of the adenoviral constructs to effect regulatable expression of hGH in vivo, we infected C57 black 6 mice with 1×10⁹ infectious viral particles by tail vein injection. To investigate the time period between viral infection and hGH expression, mice received intraperitoneal injections of RU486 over a period of 2 weeks after a single tail vein injection of the virus. As shown in (FIG. 3A), serum hGH is not detectable until day 8. At day 10 post-viral infection hGH is detectable and the levels increase sharply. At day 14, up to 10 μg/ml hGH is detectable in the serum (50,000-fold induction). The transgene expression is undetectable in the absence of the ligand. These results indicate that optimal RU486 inducible hGH expression is achieved 2 weeks after infection with the viral constructs.

### Example 5

### Kinetics of Induction of hGH Gene Expression

To investigate the kinetics of the regulatory system, mice received a single RU486 administration two weeks after the initial infection and serum hGH levels were measured at different time intervals. Three hours after administration of the drug, hGH levels are detectable in the serum of the animals (FIG. 3B). A maximum level of hGH is observed 12 hours after RU486 administration. It decreases to low hGH serum levels at 120 hours and is undetectable at 192 hours. This decline of hGH expression correlates well with the metabolism of RU486 in the mice. In contrast to the slow kinetics of hGH expression observed directly after the initial viral infection (FIG. 3A), the antiprogestin-mediated induction of hGH in these mice (FIG. 3B) is rapid and can be detected within hours.

### Example 6

### Repetitive Induction of hGH Expression

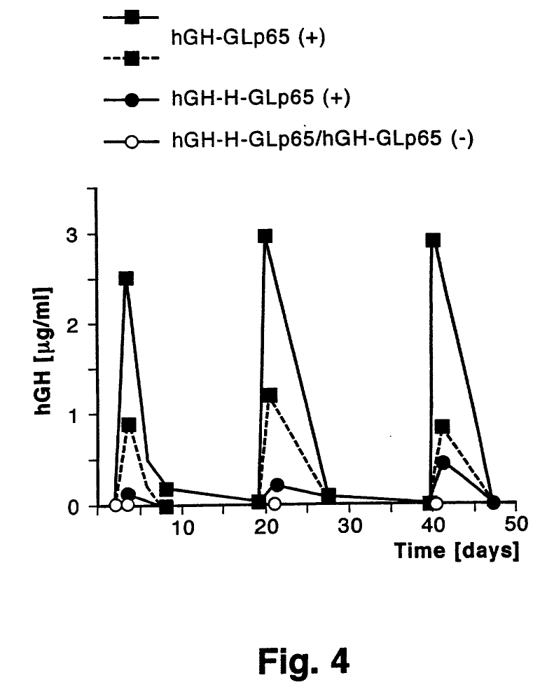

To examine if hGH expression could be reinduced, an identical dose of RU486 was administered at multiple time points to mice infected with regulatable adenoviral vectors. Mice receiving multiple RU486 administrations could be repeatedly induced over an extended period of time (FIG. 4). Twelve hours after a single administration of RU486 (250 μg/kg) a strong induction of hGH (2.5 μg/ml) is detected and over time these levels decline until hGH serum levels are no longer detectable. Similar expression levels of hGH could be obtained by repeated administration of the drug (250 μg/kg), whereas mice receiving only sesame oil had no detectable hGH serum levels. Another group of experimental animals could be reinduced up to 5 times over a period of 12 weeks. This group of animals responded equally well to oral RU486 administration with comparable hGH levels. These results demonstrate that by infecting mice with our regulatable adenoviral vector, a transgene can be induced multiple times to the same extent upon RU486 administration in vivo.

### Example 7

### Insulator Effect on hGH Expression

FIG. 4 also shows the in vivo effect of an insulator sequence when combined with an adenoviral vector. Both adenoviral vectors hGH-GLp65 (no insulator) and hGH-H-GLp65 (with insulator) are RU486 inducible and show similar kinetics after viral infection. A possible difference between the two vectors is the expression level of the transgene. As the graph shows, hGH-GLp65 seems to have a higher expression level of hGH compared to the vector containing the insulator sequences (hGH-H-GLp65). This finding was consistently observed in all experiments presented.

Mice infected with hGH-GLp65 consistently exhibited higher transgene expression levels than mice infected with the vector harboring the insulator. In contrast, the data we have obtained when transducing hepatic cell lines show similar expression levels with the two vectors. In addition, both adenoviral vectors show no detectable expression levels of hGH in the absence of RU486 in vivo, whereas in cell culture hGH-GLp65 shows low basal hGH expression. Thus, using our regulatable adenoviral vector, a difference between infection of cultured cells and experimental mice can be observed.

### Example 8

### Physiological Effect of hGH after Prolonged Expression

To achieve expression of hGH over a longer period of time, adenoviral infected mice received biodegradable pellets containing RU486 introduced by subcutaneous implanting. Blood was drawn from these animals at indicated time points after implantation. Since it is known that constitutive expression of hGH in mice leads to growth stimulation (Palmiter, R. D., et al., Science, 222:809-14, 1983), the weight of the animals was also monitored to show the physiological effect of the induced protein.

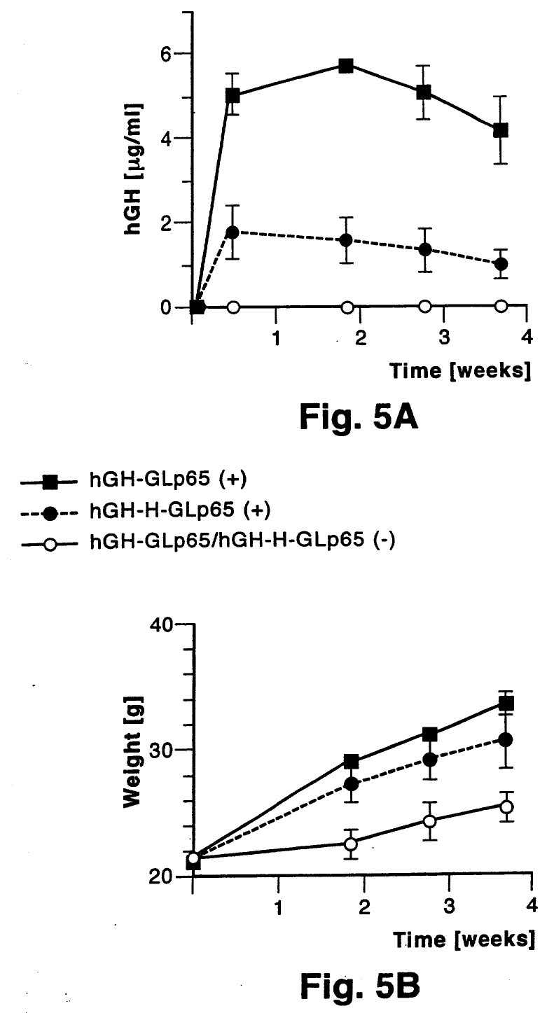

Mice receiving the RU486 pellet expressed hGH over a prolonged period of time, whereas animals receiving only the carrier showed no detectable amounts of hGH (FIG. 5A). This data correlates with the weight gain seen for the mice receiving RU486 (FIG. 5B). At day 3 after RU486 administration, the mice showed hGH levels of up to 5 μg/ml. Over the next ten days expression levels rose to a concentration of 6 μg/ml. This hGH expression was monitored for up to 4 weeks.

In response to the high levels of growth hormone expression, mice increased in weight by up to 60% within this time period. However, adenoviral infected mice treated with carrier showed only a slight increase in weight. Over the time span of 4 weeks, hGH levels decreased very slightly in the animals. This is anticipated since hGH has been shown to be immunogenic in mice (Potter, M. A., Hum Gene Ther, 9:1275-82, 1998), and this marginal decrease in hGH could be due to neutralizing antibodies raised against the protein. When hGH was induced multiple times over a short period of time we were able to express the transgene repeatedly to the same extent for up to 2 months (FIG. 4). This experiment again shows that mice infected with adenoviral constructs harboring the insulator sequence have significantly lower hGH expression levels than when the infection was performed with the vector lacking this sequence.

Conclusion

The above example applications, relating to the present invention, should not, of course, be construed as limiting the scope of the invention. Such variations of the invention, now known or later developed, which would fall within the purview of those skilled in the art are to be considered as falling within the scope of the invention as hereinafter claimed.

All patents and publications mentioned in the specification are hereby incorporated by reference to the same extent as if each individual publication was specifically and individually indicated to be incorporated by reference.

1. An adenoviral vector comprising a coding sequence for a modified steroid hormone receptor protein, wherein said receptor protein is capable of activating transcription of a desired gene when in the presence of an agonist for the receptor protein and when bound to an antagonist for the receptor protein.

2. The adenoviral vector of claim 1, wherein said modified steroid hormone receptor ligand binding domain by deletion of carboxy terminal amino acids.

3. The adenoviral vector of claim 2, wherein said deletion of said carboxy terminal amino acids comprises deletion of from about 1 to about 120 amino acids.

4. The adenoviral vector of claim 3, wherein said deletion of said carboxy terminal amino acids comprises deletion of from about one to about 60 amino acids.

5. The adenoviral vector of claim 4, wherein said deletion of carboxy terminal amino acids comprises deletion of 42 amino acids.

6. The adenoviral vector of claim 1, wherein the modified steroid hormone receptor protein comprises a modified ligand binding domain.

7. The adenoviral vector of claim 6, wherein the modified mutated ligand binding domain of said modified steroid hormone receptor protein is modified to bind a compound selected from the group consisting of non-natural ligands, anti-hormones and non-native ligands.

8. The adenoviral vector of claim 7, wherein said non-natural ligand is RU486.

9. The adenoviral vector of claim 7, wherein the ligand binding domain of said modified steroid hormone receptor protein binds a compound selected from the group consisting of 5-alpha-pregnane-3,2-dione; 11β-(4-dimethylaminophenyl)-17β-hydroxy-17α-propinyl-4,9-estradiene-3-one; 11β-(4-dimethylaminophenyl)-17α-hydroxy-17β-(3-hydroxypropyl)-13α-methyl-4,9-gonadiene-3-one; 11β-(4-acetylphenlyl)-17β-hydroxy-17α-(1-propinyl)-4,9-estradiene-3-one; 11β-(4-dimethylaminophenyl)-17β-hydroxy-17α-(3-hydroxy-1(Z)-propenylestra-4,9-diene-3-one; (7β, 11β, 17β)-11-(4-dimethylaminophenyl)-7-methyl-4′,5′-dihydrospiro[ester-4,9-diene-17,2′(3′H)-furan]-3-one; (11β, 14β, 17α)-4′,5′-dihydro-11-(4-dimethylaminophenyl)-[spiroestra-4,9-diene-17,2′(3′H)-furan]-3-one.

10. The adenoviral vector of claim 6, wherein said modified ligand binding domain is modified by deletion of about 2-5 carboxyl terminal amino acids from the ligand binding domain.

11. The adenoviral vector of claim 1, wherein the modified steroid hormone receptor protein comprises a non-native or modified DNA binding domain.

12. The adenoviral vector of claim 11, wherein the non-native or modified DNA binding domain is selected from the group consisting of GAL-4 DNA, virus DNA binding site, insect DNA binding site and a non-mammalian DNA binding site.

13. The adenoviral vector of claim 1 wherein said modified steroid hormone receptor protein further comprises a transactivation domain selected from the group consisting of VP-16, TAF-1, TAF-2, TAU-1 and TAU-2 linked to the modified steroid receptor.

14. The adenoviral vector of claim 1, wherein the modified steroid hormone receptor protein is a progesterone receptor with the ligand binding domain replaced with modified ligand binding domain which binds non-natural or non-native ligands.

15. The adenoviral vector of claim 1, wherein said modified steroid hormone receptor is tissue specific.

16. The adenoviral vector of claim 15, wherein the tissue specificity of said modified steroid hormone receptor is determined by adding a transactivation domain which is specific to a given tissue.

17. The adenoviral vector of claim 15, wherein the tissue specificity is determined by the ligand which binds to the modified steroid hormone receptor.

18. The adenoviral vector of claim 15, wherein the modified steroid hormone receptor further comprises the addition of a tissue-specific cis-element to the target gene.

19. The adenoviral vector of claim 1, wherein said vector is capable of regulating expression of a nucleic acid cassette in a transgenic animal.

20. An adenoviral vector of claim 1 wherein the modified steroid hormone receptor regulates expression of a nucleic acid cassette in a plant.

21. The adenoviral vector of claim 1, wherein said vector encodes a modified glucocorticoid receptor protein.

22. The adenoviral vector of claim 1, wherein said vector comprises:
a coding sequence for a modified steroid hormone receptor for regulating expression of a promoter transcriptionally linked to nucleic acid encoding a desired protein, said modified steroid hormone receptor comprising:
a DNA binding domain which binds said promoter;
a transactivation domain which causes transcription from said promoter when said modified steroid hormone receptor is bound to said promoter and to an agonist for said modified steroid hormone receptor; and
a modified steroid hormone superfamily receptor ligand binding domain distinct from a naturally occurring steroid hormone superfamily receptor ligand binding domain in that when bound to an antagonist for said naturally occurring steroid hormone superfamily receptor said modified steroid hormone receptor activates said transactivation domain to cause said transcription of said nucleic acid.

23. The adenoviral vector of claim 1, wherein said vector further comprises an insulator sequence.

24. The adenoviral vector of claim 1, wherein the adenoviral vector is capable of accepting a large insert.

25. The adenovial vector of claim 1, wherein said adenoviral vector does not encode a virus.

26. A transgenic animal whose cells contain an adenoviral vector of claim 1.

27. A transfected cell containing DNA which codes for the modified steroid hormone receptor protein of claim 1.

28. The transfected cell of claim 27, wherein said cell is selected from the group consisting of yeast, mammalian and insect cells.

29. The transfected cell of claim 28, wherein said cell is the yeast Saccharomyces cerevisiae.

30. The transfected cell of claim 28, wherein said cell is a mammalian cell selected from the group consisting of HeLa, CV-1, COSM6, HepG2, CHO and Ros 17.2

31. The transfected cell of claim 28, wherein said cell is an insect cell selected from the group consisting of SF9, Drosophila, butterfly and bee.

32. A composition of matter comprising a coding sequence for a modified steroid hormone receptor protein of claims 1 linked to a nucleic acid cassette, said coding sequence and said nucleic acid cassette being contained in an adenoviral vector, wherein said cassette/modified steroid hormone receptor complex is positionally and sequentially oriented in said vector such that the nucleic acid in the cassette can be transcribed and when necessary translated in a target cell.

33. The composition of matter of claim 32 comprising a promoter which contains steroid response elements.

34. A method of making a transformed cell in situ comprising the step of contacting said cell with an adenoviral vector of claim 1 for sufficient time to transform said cell, wherein said transformed cell expresses a modified glucocorticoid receptor protein encoded by said vector.

35. A method for regulating expression of a nucleic acid cassette in gene therapy comprising the step of attaching the coding sequence of the modified steroid receptor protein of claim 1, to a nucleic acid cassette to form a nucleic acid cassette/modified steroid receptor protein complex for use in the gene therapy and inserting said complex into an adenoviral vector.

36. The method of claim 35 further comprising the step of administering a pharmacological dose of the adenoviral vector to an animal or human to be treated.

37. The method of claim 35 for regulating expression of a nucleic acid cassette in gene therapy, wherein the nucleic acid cassette and the modified steroid receptor protein are in a positional relationship such that the expression of the nucleic acid sequence in the nucleic acid cassette is capable of being up-regulated or down-regulated by the modified steroid receptor protein.

38. The method of claim 35 for treating a disease wherein the nucleic acid cassette contains the nucleic acid sequence coding for a protein selected from the group consisting of a glucocorticoid receptor protein, a hormone, a neurotransmitter and a growth factor.

39. The method of claim 38, further comprising the step of encapsulating the brain cell containing the nucleic acid cassette/modified steroid hormone receptor complex in a permeable structure, said permeable structure capable of allowing the passage of activators of the modified steroid hormone receptor and protein translated from the nucleic acid sequence but preventing passage of immune cells.

40. The method of claim 39, wherein the nucleic acid sequence is transcribed to produce a protein after the animal or human is given a pharmacological dose of an anti-progesterone.

41. The method of claim 40, wherein the amount of protein produced in the transformed cell is proportional to the dose of anti-progesterone.

42. The method of claim 35, wherein the coding sequence for the modified steroid hormone receptor and the nucleic acid cassette are in separate adenoviral vectors and are co-injected into a target cell or animal.

43. The method of claim 35, wherein the nucleic acid cassette expression is regulated in a transgenic animal.

44. The method of claim 35, wherein nucleic acid cassette expression is regulated in a plant.

45. The method of claim 35, wherein said regulation is transactivation of glucocorticoid responsive genes.

46. The method of claim 35, wherein said regulation is transrepression of NF_{κ}-B and AP-1 regulated genes.

47. A method of using a modified steroid receptor protein comprising the steps of transforming a cell with an adenoviral vector of claim 1, wherein said transformed cells express said modified steroid receptor protein and said modified steroid receptor protein is capable of regulating the expression of steroid responsive genes by binding a non-natural ligand.

48. The method of claim 47, wherein said transformed cell is selected from the group consisting of a liver cell, a brain cell, a muscle cell, lung cell and a synovial cell.

49. The method of claim 38, wherein said disease is selected from the group consisting of arthritis, asthma, senile dementia, Parkinson's disease, growth hormone insufficiency, aging disorders, obesity, low hematocrit, low vascularization of cardiac or peripheral muscle, hypercholesterolemia, hemophilia, and cancer.


SEQUENCE LISTING (1 sequence)

SEQ ID NO: 1 - DNA, 19 bp, Artificial Sequence
  A DNA linker for ligating to DNA fragment(s) and generating a Sal I restriction enzyme cleavage site.
tcgacgagatatcaagcag